(19) United States
(12) Patent Application Publication
(10) Pub. No.: US 2022/0387722 A1
(43) Pub. Date: Dec. 8, 2022

(54) INJECTION PEN DEVICES, SYSTEMS, AND METHODS FOR MEDICINE ADMINISTRATION AND TRACKING

(71) Applicant: Medtronic MiniMed, Inc., Northridge, CA (US)

(72) Inventors: Michael R. Mensinger, San Diego, CA (US); Ellis Garai, Woodland Hills, CA (US); Eric A. Larson, Simi Valley, CA (US)

(21) Appl. No.: 17/342,443

(22) Filed: Jun. 8, 2021

(51) Int. Cl. A61M 5/315; A61M 5/31; G16H 20/17

(52) U.S. Cl. CPC A61M 5/31568; A61M 5/3146; A61M 5/31546; A61M 5/3157; G16H 20/17; A61M 2005/3126; A61M 2205/52; A61M 2205/583; A61M 2205/3331

(57) ABSTRACT

A method for medicine administration and tracking and system for the same that may implement the method via a health management application running on a computing device includes tracking movement of an injection pen from a start location to a dispensing location, detecting a dispensing event of medicine dispensed from the injection pen at the dispensing location, determining a location of the dispensing location relative to a user's body, associating information relating to the detected dispensing event with information relating to the determined location, and providing feedback based upon the detected dispensing event information and the determined location information.

### FIELD

The present disclosure relates to devices, systems, and methods for medicine administration and tracking and, more specifically, to injection pen devices, systems, and methods for medicine administration and tracking configured to differentiate between priming and injection actions and/or to provide information relating to a medicine dispensing event.

### BACKGROUND

Diabetes mellitus (“diabetes”) is a metabolic disease associated with high blood sugar due to insufficient production or use of insulin by the body. Diabetes affects hundreds of millions of people and is among the leading causes of death globally. Diabetes has been categorized into three types: type 1, type 2, and gestational diabetes. Type 1 diabetes is associated with the body's failure to produce sufficient levels of insulin for cells to uptake glucose. Type 2 diabetes is associated with insulin resistance, in which cells fail to use insulin properly. Gestational diabetes can occur during pregnancy when a pregnant woman develops a high blood glucose level. Gestational diabetes often resolves after pregnancy; however, in some cases, gestational diabetes develops into type 2 diabetes.

Various diseases and medical conditions, such as diabetes, require a user to self-administer doses of medicine. When administering a liquid medicine by injection, for example, the appropriate dose amount is set and then dispensed by the user, e.g., using a syringe, a medicine delivery pen, or a pump. Regardless of the particular device utilized for injecting the liquid medicine, it is important to accurately track the medicine dosed, particularly for managing lifelong or chronic conditions like diabetes.

### SUMMARY

To the extent consistent, any of the aspects and features detailed herein can be utilized with any of the other aspects and features detailed herein in any suitable combination.

Provided in accordance with aspects of the present disclosure is a method for medicine administration and tracking including tracking movement of an injection pen from a start location to a dispensing location, detecting a dispensing event of medicine dispensed from the injection pen at the dispensing location, determining a location of the dispensing location relative to a user's body, associating information relating to the detected dispensing event with information relating to the determined location, and providing feedback based upon the detected dispensing event information and the determined location information.

In aspects, the dispensing location relative to a user's body may be a proximity to the user's body, a proximity to a portion (or one of several portions) of the user's body, a particular location on the user's body, etc.

In an aspect of the present disclosure, the feedback includes a recommended injection location. The recommended injection location may be determined based upon evaluating at least one physiological parameter, e.g., insulin absorbed or an insulin absorption rate, associated with the dispensing event at the determined location.

In another aspect of the present disclosure, the feedback includes a recommendation to change an injection location. The recommendation to change the injection location may be determined based upon evaluating a number of dispensing events occurring at the determined location.

In yet another aspect of the present disclosure, the method further includes determining whether the dispensing event is a priming event or an injection event based upon the determined location (relative to the user's body). In such aspects, the associating and providing feedback may only be performed in a case where it is determined that the dispensing event is a priming event.

A medicine administration and tracking system provided in accordance with the present disclosure includes an injection pen configured to dispense medicine and a computing device. The injection pen includes at least one sensor configured to sense a dispensing event of medicine dispensed from the pen, at least one motion sensor configured to provide information regarding movement of the injection pen, and an electronic unit configured to transmit information regarding the dispensing event and the information regarding movement of the injection pen. The computing device is configured to receive the information regarding the dispensing event and the information regarding movement of the injection pen and includes a processor and a memory storing a health management application. When the health management application is executed by the processor, the processor is caused to track movement of the injection pen from a start location to a dispensing location based upon the information regarding movement of the injection pen (wherein the dispensing location corresponds to the position where the dispensing event occurred), determine a location of the dispensing location relative to a user's body, associate the information regarding the dispensing event with information relating to the determined location, and provide feedback based upon the dispensing event information and the determined location information.

In an aspect of the present disclosure, the feedback includes a recommended injection location determined based upon evaluating at least one physiological parameter, e.g., insulin absorbed and/or an insulin absorption rate, associated with the dispensing event at the determined location.

In another aspect of the present disclosure, the feedback includes a recommendation to change an injection location determined based upon evaluating a number of dispensing events occurring at the determined location.

In still yet another aspect of the present disclosure, the processor is further caused to determine whether the dispensing event is a priming event or an injection event based upon the determined location and to associate the information and provide the feedback only in a case where it is determined that the dispensing event is a priming event.

A medicine injection pen provided in accordance with aspects of the present disclosure includes a body, a drive member disposed within the body and configured to move relative to the body upon actuation of the drive member, a cartridge housing engaged with the body and configured to retain a medicine cartridge therein, at least one sensor, and a thin-film pressure sensor. The medicine cartridge includes a medicine vial configured to retain a liquid medicine therein, a needle disposed at a dispensing end of the medicine vial, and a piston configured to slide within the medicine vial. The drive member is configured to move relative to the body upon actuation thereof to urge the piston to slide within the medicine vial to thereby dispense at least some of the liquid medicine through the needle. The at least one sensor is configured to sense movement of the drive member relative to the body upon actuation of the drive member to enable determination of an amount of the liquid medicine dispensed based on the sensed movement. The thin-film pressure sensor is disposed on one of: a portion of the cartridge housing, a portion of the medicine vial, or a portion of needle and configured to detect pressure applied by a user's skin to thereby enable determination of whether the amount of the liquid medicine dispensed was dispensed as a priming event or an injection event.

In an aspect of the present disclosure, the thin-film pressure sensor is an ultra thin-film pressure sensor and/or defines a ring-shaped configuration.

In aspects of the present disclosure, the thin-film pressure sensor is disposed on an exterior end face of the cartridge housing, an exterior end face of a hub of the needle, or on the medicine vial.

In aspects of the present disclosure, mating contacts are disposed on the hub of the needle and the cartridge housing, the medicine vial and the cartridge housing, and/or between the cartridge housing and the body to electrically connect the thin-film pressure sensor to an electronics unit disposed within the body.

Another medicine administration and tracking system provided in accordance with the present disclosure includes an injection pen configured to dispense medicine. The injection pen includes at least one sensor configured to sense a dispensing event of medicine dispensed from the pen, at least one motion sensor configured to provide information regarding an orientation of the injection pen, and an electronic unit configured to transmit information regarding the dispensing event and the information regarding the orientation of the injection pen. The computing device is configured to receive the information regarding the dispensing event and the information regarding the orientation of the injection pen. The computing device includes a processor and a memory storing a health management application that, when executed by the processor, causes the processor to determine, based on the information regarding the dispensing event, occurrence of the dispensing event; determine, based on the information regarding the orientation of the injection pen, the orientation of the injection pen at a time of the dispensing event; determine, based on the orientation of the injection pen at the time of the dispensing event, whether the dispensing event is a priming event or an injection event; and provide feedback regarding whether the dispensing event was as priming event or an injection event.

### BRIEF DESCRIPTION OF DRAWINGS

FIG. 1A is a schematic illustration of a medicine administration and tracking system provided in accordance with the present disclosure including a medicine injection pen, a computing device, and, in aspects, a sensor device and/or a data processing system;

FIG. 1B is a block diagram of the medicine injection pen of the system of FIG. 1A;

FIG. 1C is a block diagram of the computing device of the system of FIG. 1A;

FIGS. 2A and 2B are perspective and longitudinal, cross-sectional views, respectively, of the medicine injection pen of FIG. 1B;

FIGS. 3A and 3B are side, partial longitudinal, cross-sectional views of the medicine injection pen of FIG. 1B with the medicine cartridge in a full condition and the medicine cartridge in a partially emptied condition, respectively;

FIGS. 4A-4C are enlarged, longitudinal, cross-sectional views of the dispensing end of medicine injection pen of FIG. 1B including various pressure sensors in accordance with the present disclosure;

FIG. 5 is a top view of a pressure sensor configured for use as the pressure sensors of FIGS. 4A-4C;

FIGS. 6A and 6B are enlarged, longitudinal, cross-sectional views of the dispensing end of medicine injection pen of FIG. 1B including other sensors in accordance with the present disclosure;

FIGS. 7A and 7B are enlarged, longitudinal, cross-sectional views of the dispensing end of medicine injection pen of FIG. 1B including a shroud in accordance with the present disclosure disposed in extended and retracted positions, respectively;

FIGS. 8A and 8B are enlarged, longitudinal, cross-sectional views of the dispensing end of medicine injection pen of FIG. 1B including another shroud in accordance with the present disclosure disposed in extended and retracted positions, respectively;

FIG. 9A is a side, partial longitudinal, cross-sectional view of the medicine injection pen of FIG. 1B including a force sensor associated with the drive screw in accordance with the present disclosure;

FIG. 9B is a side, partial longitudinal, cross-sectional view of the medicine injection pen of FIG. 1B including a force sensor associated with the interface between the body of the pen and the cartridge housing of the pen in accordance with the present disclosure;

FIG. 10 is an enlarged, longitudinal, cross-sectional view of the actuation end of medicine injection pen of FIG. 1B including a force sensor associated with the actuation button in accordance with the present disclosure;

FIG. 11 is a side, partial longitudinal, cross-sectional view of the medicine injection pen of FIG. 1B including another sensor in accordance with the present disclosure;

FIG. 12 is a flowchart illustrating steps of a method in accordance with the present disclosure; and

FIG. 13 is a flowchart illustrating steps of another method in accordance with the present disclosure.

### DETAILED DESCRIPTION

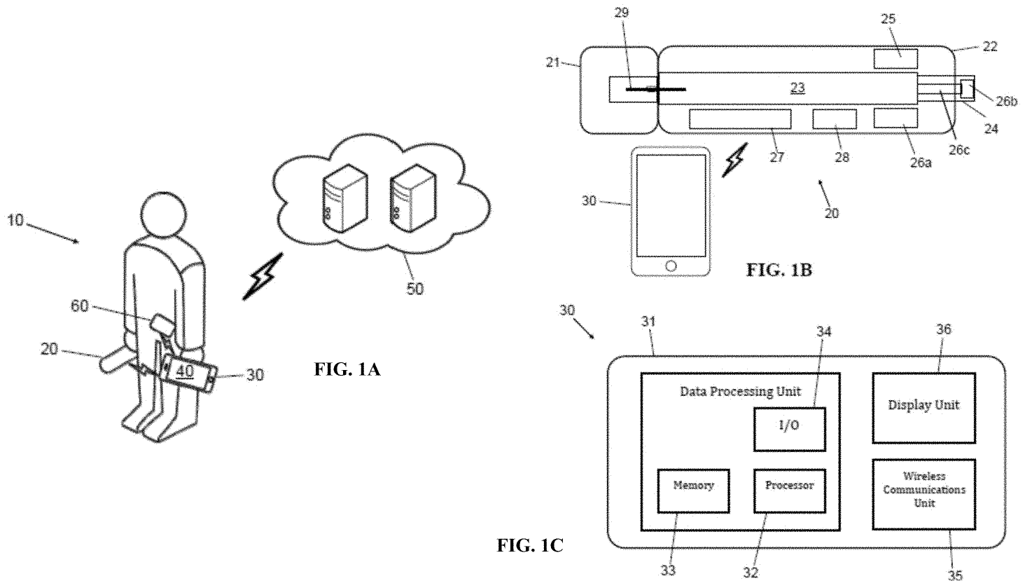

FIG. 1A illustrates a medicine administration and tracking system 10 provided in accordance with the present disclosure including a medicine injection pen 20 in wireless communication with a computing device 30 running a health management application 40 associated with pen 20 and/or other devices that are part of or connected to system 10. System 10, in aspects, further includes a data processing system 50 and/or a sensor device 60. While the reusable injection pens with replaceable cartridges of the present disclosure are detailed herein configured for use as medicine injection pen 20 of system 10 with respect to diabetes management, it is understood that the reusable injection pens of the present disclosure are also applicable to management of other diseases and medical conditions and/or for use with other medicine administration and tracking systems.

Medicine injection pen 20, described in greater detail below, is a reusable injection pen configured to removably receive a medicine cartridge, e.g., a cartridge of insulin, for injecting a selected dose of insulin into a user and recording information concerning the injected dose of insulin, e.g., a dose amount and/or timestamp data associated with the dose.

Computing device 30 is detailed and illustrated herein as a smartphone, although any other suitable computing device may be provided such as, for example, a tablet, a wearable computing device (e.g., a smart watch, smart glasses, etc.), a laptop and/or desktop computer, a smart television, a network-based server computer, etc.

Health management application 40 is paired with pen 20, which may be a prescription-only medical device, via smartphone 30, although other suitable configurations are also contemplated. In aspects, the pairing of smartphone 30 with pen 20 at least partially unlocks health management application 40 to enable the user to utilize some or all features of health management application 40, e.g., according to the user's prescription. Thus, the act of pairing can unlock and enable the functionality of health management application 40 and/or system 10 (including pen 20), while health management application 40 (and/or system 10) may provide only limited features in the absence of pairing with pen 20.

Health management application 40 of smartphone 30, in aspects, can monitor and/or control functionalities of pen 20 and provide a dose calculator module and/or decision support module that can calculate and recommend a dose of medicine for the user to administer using pen 20. Health management application 40 provides a user interface, on the user interface of smartphone 30, to allow a user to manage health-related data. For example, health management application 40 can be configured to control some functionalities of pen 20 and/or to provide an interactive user interface to allow a user to manage settings of pen 20 and/or settings for smartphone 30 that can affect the functionality of system 10 (FIG. 1A). Smartphone 30 can additionally or alternatively be used to obtain, process, and/or display contextual data that can be used to relate to the health condition of the user, including the condition for which pen 20 is used to treat. For example, smartphone 30 may be operable to track the location of the user; physical activity of the user including step count, movement distance and/or intensity, estimated calories burned, and/or activity duration; and/or interaction pattern of the user with smartphone 30. In aspects, health management application 40 can aggregate and process the contextual data to generate decision support outputs, e.g., on the user interface, to guide and aid the user in monitoring their condition, using pen 20, and/or managing their behavior to promote treatment and better health outcomes.

In aspects, system 10 further includes a data processing system 50 in communication with pen 20 and/or smartphone 30. Data processing system 50 can include one or more computing devices in a computer system and/or communication network accessible via the internet, e.g., including servers and/or databases in the cloud. System 10 can additionally or alternatively include sensor device 60 to monitor one or more health metrics and/or physiological parameters of the user. Examples of health metric and physiological parameter data monitored by sensor device 60 include analytes (e.g., glucose), heart rate, blood pressure, user movement, temperature, etc. Sensor device 60 may be a wearable sensor device such as a continuous glucose monitor (CGM) to obtain transcutaneous or blood glucose measurements that are processed to produce continuous glucose values. For example, the CGM can include a glucose processing module implemented on a stand-alone display device and/or implemented on smartphone 30, which processes, stores, and displays the continuous glucose values for the user. Such continuous glucose values can be utilized by health management application 40, for example, for displaying health data, in dose calculation and/or decision support, etc.

With reference to FIG. 1B, pen 20 includes a cap 21 configured to protect a medicine dispensing element (e.g., a needle 29) and a body 22 configured to contain a replaceable medicine cartridge 23, e.g., an insulin cartridge. Pen 20 further includes a dose dispensing mechanism 24 to dispense (e.g., deliver) medicine contained in medicine cartridge 23 out of pen 20 (e.g., through needle 29); a dose setting mechanism 25 to enable the selection and/or setting of a dose of medicine to be dispensed; an operations monitoring mechanism 28 (e.g., including one or more switches, sensors (electrical, optical, acoustic, magnetic, etc.), encoders, etc.) to qualitatively determine that pen 20 is being operated and/or to monitor the operation of pen 20 (e.g., to quantitatively determine an amount of medicine set and/or dosed); and an electronics unit 27 that can include a processor, a memory, a transceiver, and a battery or other suitable power source.

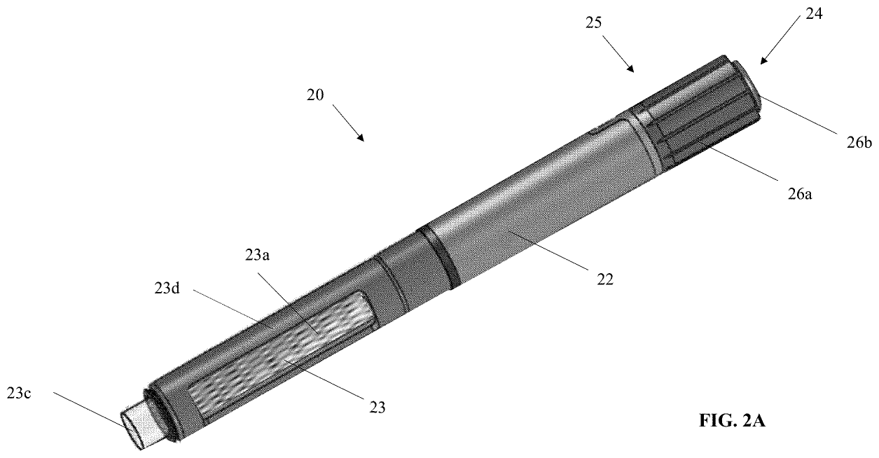

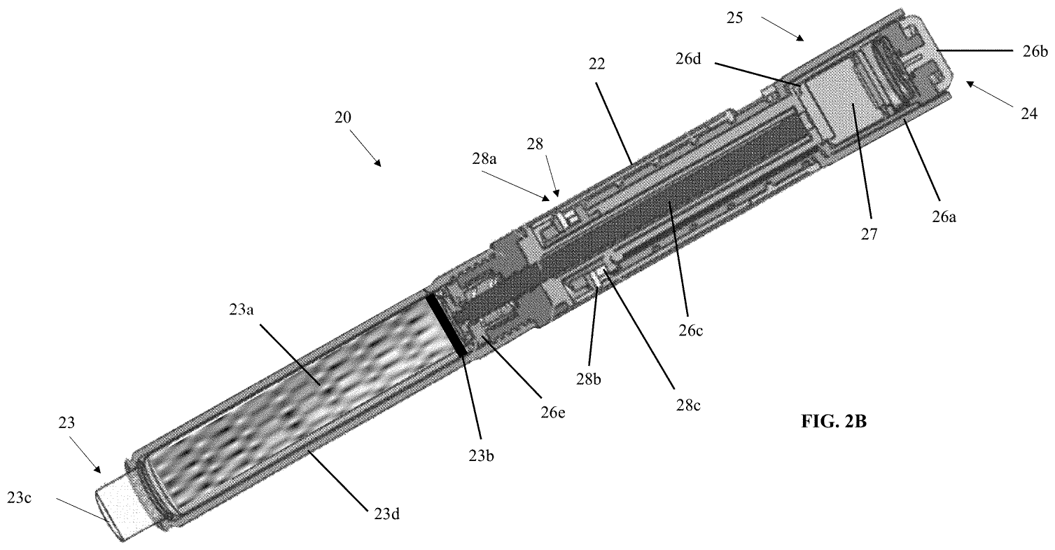

In aspects, in order to operate pen 20, the user first sets e.g., dials, a dose using a dose knob 26a of dose setting mechanism 25. For example, the dose may be adjusted up or down to achieve a desired dose amount prior to administration of the dose by rotating dose knob 26a in an appropriate direction. Once the appropriate dose has been set, the user applies a force against a dose dispensing button 26b of dose dispensing mechanism 24 to begin dispensing. More specifically, to begin dispensing, the user presses against the portion of dose dispensing button 26b that protrudes from body 22 of pen 20 to thereby drive a driving element 26c, e.g., a drive screw 26c, of dose dispensing mechanism 24 against an abutment, e.g., piston 23b (FIG. 2B), of medicine cartridge 23 to dispense an amount of medicine from cartridge 23 through needle 29 into the user in accordance with the dose amount set by dose setting mechanism 25, e.g., dose knob 26a, during setting.

Operations monitoring mechanism 28 of pen 20 senses movement of a rotating and/or translating driving component (e.g., drive screw 26c (see also FIG. 2B)) of dose dispensing mechanism 24. Operations monitoring mechanism 28 may include one or more switches, sensors, and/or encoders for this purpose. More specifically, any suitable switch(es), sensor(s), and/or encoder(s) may be utilized to sense rotary and/or linear movement. Non-limiting examples of such include rotary and linear encoders, Hall effect and other magnetic-based sensors, linearly variable displacement transducers, optical sensors, etc. With respect to an encoder, for example, the encoder can be configured to sense the rotation of drive screw 26c (FIG. 2B) that, in turn, translates to dispense medicine; thus, by sensing rotation of drive screw 26c (FIG. 2B), the translational movement of drive screw 26c can be readily determined. Movement of the encoder may be detected as data processed by the processor of electronics unit 27 of pen 20, from which the amount of medicine dosed can be determined.

In aspects, the processor of electronics unit 27 of pen 20 can store the dose along with a timestamp for that dose and/or any other information associated with the dose. In aspects, the transceiver of electronics unit 27 enables pen 20 to transmit the dose and related information to smartphone 30. In such aspects, once the dose is transmitted, the dose data and any related information associated with that particular transmitted dose is marked in the memory of electronics unit 27 of pen 20 as transmitted. If the dose is not yet transmitted to smartphone 30 such as, for example, because no connection between the pen 20 and smartphone 30 is available, then the dose and associated data can be saved and transmitted the next time a successful communication link between pen 20 and smartphone 30 is established.

The timestamp may be the current time or a time from a count-up timer. When the dose and associated information is communicated to health management application 40 running on smartphone 30, the timestamp and/or “time-since-dose” parameter (as determined by the count-up timer) is transmitted by pen 20 and received by smartphone 30 for storage in memory 33 of data processing unit 31 of the smartphone 30 (see FIG. 1C). Where a count-up timer is utilized, the time of the dose can be determined without pen 20 having to know the current time, which can simplify operation and setup of pen 20. That is, health management application 40 can determined the time of dose based on the current time and the value returned from the count-up timer.

Dose dispensing mechanism 24 of pen 20 can include a manually powered mechanism (user powered and/or mechanically biased), a motorized mechanism, or an assisted mechanism (e.g., a mechanism that operates partly on manual power and partly on motorized power). Regardless of the particular configuration of the dose dispensing mechanism 24, as noted above, when a force (e.g., a manual force, electrically-powered motor force, or combinations thereof) is applied to drive screw 26c of dose dispensing mechanism 24, drive screw 26c turn provides a force to urge medicine from medicine cartridge 23 to deliver the set or dialed dose. In aspects, dose dispensing mechanism 24 can be operated such that rotation and/or translation of the driving element, e.g., drive screw 26c, is facilitated by a variable tension spring or a variable speed motor to inject the dose over a specific time frame (e.g., 1 s, 5 s, etc.) to help reduce the pain of dosing and/or for other purposes.

FIG. 1C illustrates smartphone 30 of system 10 (FIG. 1A) including a data processing unit 31, a wireless communications unit 35, and a display unit 36. Data processing unit 31 includes a processor 32 to process data, a memory 33 in communication with the processor 32 to store data, and an input/output unit (I/O) 34 to interface processor 32 and/or memory 33 to other modules, units, and/or devices of smartphone 30 and/or external devices. Processor 32 can include a central processing unit (CPU) or a microcontroller unit (MCU). Memory 33 can include and store processor-executable code, which when executed by processor 32, configures the data processing unit 31 to perform various operations, e.g., such as receiving information, commands, and/or data, processing information and data, and transmitting or providing information/data to another device. In aspects, data processing unit 31 can transmit raw or processed data to data processing system 50 (FIG. 1A). To support various functions of data processing unit 31, memory 33 can store information and data, such as instructions, software, values, images, and other data processed or referenced by processor 32. For example, various types of Random Access Memory (RAM) devices, Read Only Memory (ROM) devices, Flash Memory devices, and other suitable storage media can be used to implement storage functions of memory 33. I/O 34 of data processing unit 31 can interface data processing unit 31 with wireless communications unit 35 to utilize various types of wired or wireless interfaces compatible with typical data communication standards, for example, which can be used in communications of data processing unit 31 with other devices such as pen 20, via a wireless transmitter/receiver (Tx/Rx), e.g., including, but not limited to, Bluetooth, Bluetooth low energy, Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE cellular communication methods, NFC (Near Field Communication), and parallel interfaces. I/O 34 of data processing unit 31 can also interface with other external interfaces, sources of data storage, and/or visual or audio display devices, etc. to retrieve and transfer data and information that can be processed by processor 32, stored in memory 33, and/or exhibited on an output unit of smartphone 30 and/or an external device. For example, display unit 36 of smartphone 30 can be configured to be in data communication with data processing unit 31, e.g., via I/O 34, to provide a visual display, an audio display, and/or other sensory display that produces the user interface of the health management application 40 (FIG. 1A). In some examples, display unit 36 can include various types of screen displays, speakers, or printing interfaces, e.g., including but not limited to, light emitting diode (LED), or liquid crystal display (LCD) monitor or screen, cathode ray tube (CRT) as a visual display; audio signal transducer apparatuses as an audio display; and/or toner, liquid inkjet, solid ink, dye sublimation, inkless (e.g., such as thermal or UV) printing apparatuses, etc.

Once smartphone 30 receives the dose and related information (e.g., which can include time information, dose setting, and/or dose dispensing information, and other information about pen 20 and/or the environment as it relates to a dosing event), smartphone 30 stores the dose related information in memory 33, e.g., which can be included among a list of doses or dosing events. In aspects, via the user interface associated with health management application 40, smartphone 30 allows the user to browse a list of previous doses, to view an estimate of current medicine active in the user's body (medicine on board, e.g., insulin on board) based on calculations performed by health management application 40, and/or to utilize a dose calculation module to assist the user regarding dose setting information on the size of the next dose(s) to be delivered. For example, the user may enter carbohydrates to be eaten and current blood sugar (which alternatively may be obtained directly from sensor device 60 (FIG. 1A)), and health management application 40 may already know insulin on board. Using these parameters, a suggested medicine dose (e.g., a recommended insulin dose), calculated by the dose determination module, may be determined. In aspects, smartphone 30 can also allow the user to manually enter dose data, e.g., boluses, which may be useful if the battery in pen 20 has been depleted or another medicine delivery device, e.g., a syringe, was utilized to dose.

Referring to FIGS. 2A and 2B, pen 20 and, in particular, the mechanical and hardware features thereof, is detailed, although other mechanical and hardware configurations of pen 20 are also contemplated. Pen 20 is shown configured as a reusable device for use with replaceable medicine cartridge 23 which, once emptied (or for other purposes), can be replaced with another medicine cartridge 23 or refilled and reinstalled for subsequent use. Medicine cartridge 23 includes a vial body 23a defining an interior volume configured to retain a volume of medicine, e.g., insulin, therein, and a piston 23b sealingly and slidingly disposed within vial body 23a such that displacement of piston 23b within vial body 23a towards the dispensing end of vial body 23a forces medicine from the interior volume through dispensing opening 23c of cartridge 23 and needle 29 (FIG. 1B) for injection into the user.

Medicine cartridge 23 is held within a cartridge housing 23d of pen 20 and, in aspects, may be seated within a corresponding cartridge adapted (not shown) positionable within cartridge housing 23d to enable use of various different medicine cartridges (e.g., of different size, shape, manufacturer, etc.) with pen 20. Cartridge housing 23d is releasably engageable with body 22 of pen 20, e.g., via threaded engagement, such that, when cartridge housing 23d is disengaged from body 22 of pen 20, medicine cartridge 23 can be removed and replaced and such that, when cartridge housing 23d is engaged with body 22 of pen 20 with a medicine cartridge 23 therein, medicine cartridge 23 is operably positioned relative to dose dispensing mechanism 24 of pen 20. However, other suitable configurations enabling removal and replacement of a medicine cartridge 23 are also contemplated.

Continuing with reference to FIGS. 2A and 2B, dose knob 26a of pen 20 may be coupled to body 22 of pen 20 in threaded engagement via corresponding threads defined on an exterior surface of a portion of dose knob 26a and an interior surface of a portion of body 22. In aspects, electronics unit 27 may reside within an electronics housing disposed or defined within dose knob 26a and be coupled thereto via a locking mechanism 26d (e.g., a catch-protrusion mechanism, a clutch, etc.) such that, when dose knob 26a is rotated into or out of body 22 to select or adjust the dose to be injected, electronics unit 27 remains stationary (e.g., wherein the locking mechanism 26d is in an unlocked state); however, when dispensing button 26b is actuated, locking mechanism 26d is engaged to lock electronics unit 27 and dose knob 26a to one another such that electronics unit 27 and dose knob 26a rotate together as they translate into body 22 upon actuation of dose dispensing mechanism 24 to inject the selected dose.

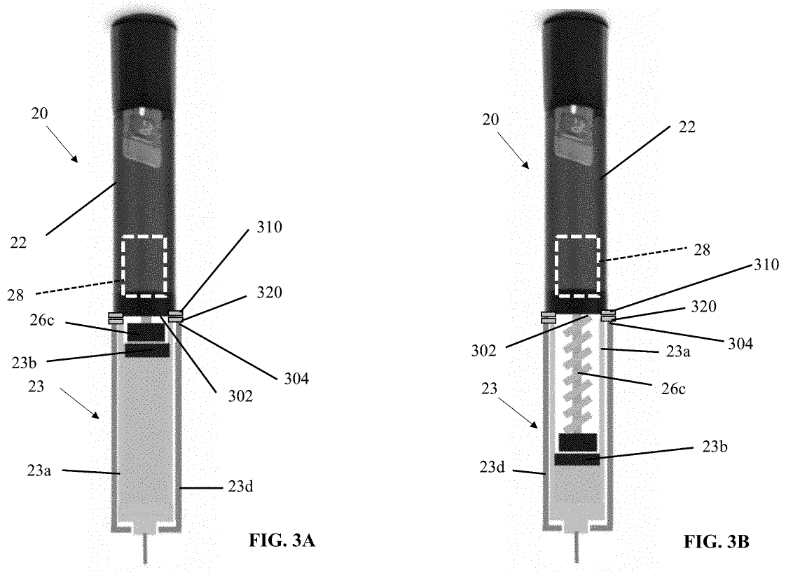

With additional reference to FIGS. 3A and 3B, the rotation of the dose knob 26a (and electronics unit 27) during actuation drives (direct or indirect) rotation of drive screw 26c which rides within a nut 26e which is fixed to body 22 of pen 20. In this manner, rotation of drive screw 26c also results in translation of drive screw 26c (due to the pitched threading of drive screw 26c) towards medicine cartridge 23 to thereby drive piston 23b through vial body 23a to expel medicine from medicine cartridge 23 for injection into the user. The extent to which dose knob 26a extends from body 22 of pen 20 prior to actuation (which corresponds to the selected dose to be injected) defines the maximum amount of rotation of dose knob 26a and, thus, drive screw 26c during actuation; as such, the amount of medicine expelled from medicine cartridge 23 during actuation cannot exceed the selected dose amount.

Operations monitoring mechanism 28 of pen 20 may include a rotary encoder 28a having a first part 28b rotationally fixed relative to body 22 of pen 20 and a second part 28c rotationally fixed relative to drive screw 26c such that relative rotation between the first and second parts 28b, 28c (which, in turn, is indicative of rotation of drive screw 26c relative to body 22 during dose dispensing), can be sensed and, thus, from which an amount of medicine dispensed can be determined (due to the proportional relationship between rotation of drive screw 26c and translation of piston 23b). Alternatively or additionally, rotary encoder 28a may be configured to sense the amount of medicine dialed for dosing. In aspects, rotary encoder 28a is an electrical contact encoder including one or more contacts disposed on one of the parts 28b, 28c and a code wheel disposed on the other part 28b, 28c, although other configurations and/or types of encoders are also contemplated. Regardless of the particular type of encoder or other sensory components of operations monitoring mechanism 28, relative motion is measured and transmitted to electronics unit 27 for processing (e.g., determining an amount of medicine dispensed), storage (e.g., storing in memory the amount of medicine dispensed together with timestamp data) and/or transmission (e.g., transmitting the stored data to smartphone 30).

Continuing with reference to FIGS. 3A and 3B, in aspects, pen 20 may include one or more contacts 310, e.g., one or more ring contacts or spaced-apart contacts, disposed on the outer periphery of body 22 of pen 20 adjacent an engagement face 302 thereof or on engagement face 302. Engagement face 302 is configured to mate with a corresponding engagement face 304 of cartridge housing 23d upon engagement, e.g., threaded engagement, of cartridge housing 23d with body 22 of pen 20. Cartridge housing 23d includes one or more contacts 320, e.g., one or more ring contacts or spaced-apart contacts, disposed on or adjacent engagement face 304. In this manner, upon engagement of cartridge housing 23d with body 22 of pen 20, contacts 310, 320 connect with one another to enable electrical communication therebetween, e.g., of power signals, control signals, data signals, sensor signals, etc. In aspects where electrical communication between cartridge housing 23d and body 22 is not required or is provided in another manner, e.g., wirelessly, contacts 310, 320 may be omitted.

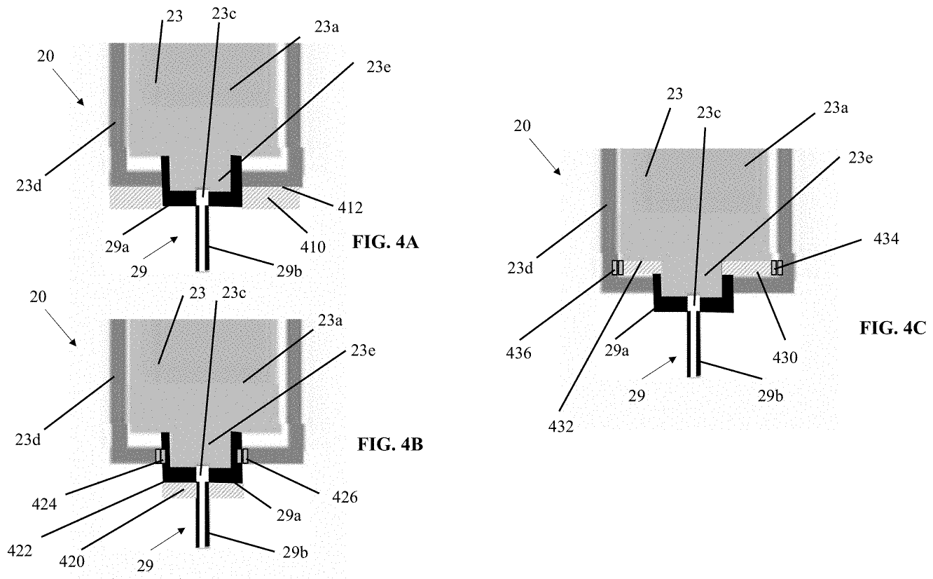

Referring generally to FIGS. 4A-12, although operations monitoring mechanism 28 (FIGS. 2B-3B), e.g., including rotary encoder 28a (FIG. 2B) or any other suitable sensor associated with movement of drive screw 26c and/or other dose delivery component, enables determination of the amount of medicine dispensed based on the rotation of drive screw 26c, operations monitoring mechanism 28 (FIG. 2B) itself cannot determine the purpose of the dispensing event, e.g., whether the dispensing event was a priming event or an injection event. That is, while the amount of medicine dispensed can be readily determined using rotary encoder 28a (FIG. 2B) (or other suitable sensor), the amount of medicine injected cannot necessarily be determined using rotary encoder 28a (FIG. 2B) without additional processing, assumptions, and/or data input. FIGS. 4A-12 provide various aspects and features of pen 20 that provide such additional processing, assumptions, and/or data input to enable the distinction between a priming event and an injection event and, thus, enabling accurate tracking of the amount of medicine injected. To the extent consistent and not explicitly contradicted below, any of the aspects and features detailed below with respect to any of the configurations of FIGS. 4A-12 can be utilized, in whole or in part, in any suitable combination in a single medicine injection device to achieve a particular purpose.

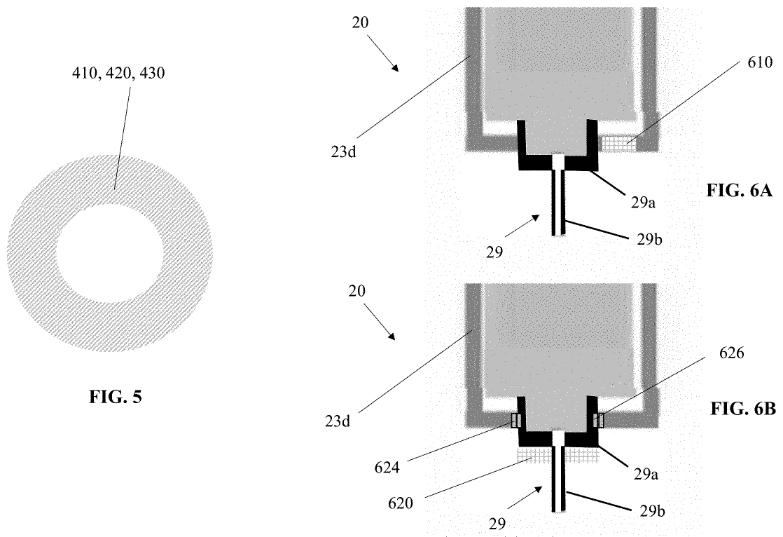

Turning to FIGS. 4A-4C and 5, a neck 23e extends from the dispensing end of vial body 23a of medicine cartridge 23 through an opening at the dispensing end of cartridge housing 23d. Thus, neck 23e is at least partially exposed to enable releasable attachment of needle 29 thereto, e.g., via threaded engagement. Needle 29 includes a needle hub 29a configured to releasably engage neck 23e and a hollow needle body 29b configured to puncture a user's skin and deliver medicine therethrough into the user's body. Upon engagement of needle hub 29a with neck 23e, dispensing opening 23c of vial body 23a is disposed in fluid communication with hollow needle body 29b to enable the delivery of medicine therethrough upon actuation of pen 20. In preparation for injection, hollow needle body 29b of needle 29 pierces the user's skin and is inserted therethrough until the skin abuts a portion of needle hub 29a and/or cartridge housing 23d. Thereafter, pen 20 can be actuated to deliver a volume of medicine, e.g., insulin, to the user.

As shown in FIGS. 4A-4C, pressure sensors 410, 420, 430 are disposed on exterior face 412 of cartridge housing 23d, exterior face 422 of needle hub 29a of needle 29, and on a face 432 of vial body 23a of cartridge 23, respectively. With additional reference to FIG. 5, pressure sensors 410, 420, 430 may be, for example, thin-film (<1.0 mm thickness) pressure sensors, ultra thin-film (<0.5 mm) pressure sensors, or any other suitable pressure sensors; may be resistance-based pressure sensors, transducer-based pressure sensors, or otherwise configured to enable detection of a magnitude of pressure applied, an applied pressure exceeding a pressure threshold, a pressure map, relative or absolute pressure trend, etc.; may be adhered to the respective face 412, 422, 432 or otherwise attached thereon; and/or may define ring-shaped configurations to enable passage of other operable components of pen 20 therethrough.

Referring in particular to FIG. 4A, with pressure sensor 410 disposed on exterior face 412 of cartridge housing 23d, pressure applied to pressure sensor 410 by the user's skin upon insertion of hollow needle body 29b of needle 29 into the user's skin until the skin abuts cartridge housing 23d can be detected, thus indicating that pen 20 is being utilized to inject a volume of medicine, e.g., insulin, into the user, rather than for a priming event. With additional reference to FIGS. 3A and 3B, pressure sensor 410 may be electrically connected, e.g., via wires (not shown) or other conductive structures extending along, within, or through cartridge housing 23d to contact(s) 320 of cartridge housing 23d which, in turn, are connected with contact(s) 310 of body 22 upon engagement of cartridge housing 23d with body 22, to thereby enable powering of pressure sensor 410 and communication of pressure signals from pressure sensor 410 to electronics unit 27 and/or operations monitoring mechanism 28.

With reference in particular to FIG. 4B, with pressure sensor 420 disposed on exterior face 422 of needle hub 29a of needle 29, pressure applied to pressure sensor 420 by the user's skin upon insertion of hollow needle body 29b of needle 29 into the user's skin until the skin abuts needle hub 29a can be detected, thus indicating that pen 20 is being utilized to inject a volume of medicine, e.g., insulin, into the user, rather than for a priming event. With additional reference to FIGS. 3A and 3B, pressure sensor 420 may be electrically connected to contacts 424 disposed on needle hub 29a of needle 29 which, in turn, connect with contacts 426 disposed on cartridge housing 23d upon engagement of needle hub 29a with neck 23e of vial body 23a when cartridge 23 is received within cartridge housing 23d. Contacts 426 are connected, e.g., via wires (not shown) or other conductive structures extending along, within, or through cartridge housing 23d to contact(s) 320 of cartridge housing 23d which, in turn, are connected with contact(s) 310 of body 22 upon engagement of cartridge housing 23d with body 22, to thereby enable powering of pressure sensor 420 and communication of pressure signals from pressure sensor 420 to electronics unit 27 and/or operations monitoring mechanism 28.

With reference in particular to FIG. 4C, with pressure sensor 430 disposed on a face 432 of via body 23a of cartridge 23, pressure applied to pressure sensor 430 by needle 29 upon insertion of hollow needle body 29b of needle 29 into the user's skin (which provides an opposite force on needle 29) can be detected, thus indicating that pen 20 is being utilized to inject a volume of medicine, e.g., insulin, into the user, rather than for a priming event. With additional reference to FIGS. 3A and 3B, pressure sensor 430 may be electrically connected to contacts 434 of via body 23a which, in turn, are electrically connected with contacts 436 of cartridge housing 23d when cartridge 23 is disposed therein. Contacts 436, in turn, are connected, e.g., via wires (not shown) or other conductive structures extending along, within, or through cartridge housing 23d to contact(s) 320 of cartridge housing 23d which, in turn, are connected with contact(s) 310 of body 22 upon engagement of cartridge housing 23d with body 22, to thereby enable powering of pressure sensor 430 and communication of pressure signals from pressure sensor 430 to electronics unit 27 and/or operations monitoring mechanism 28.

Referring back to FIGS. 3A-5, based on the data sensed by pressure sensors 410, 420, 430, it can be determined that pen 20 is being utilized to inject a volume of medicine, e.g., insulin, into the user, rather than for a priming event. More specifically, where a pressure above a pressure threshold, a particular pressure map, a particular pressure trend, and/or a duration of pressure application, etc. is measured prior to, during, or otherwise in relation to actuation of pen 20 to dispense medicine, the amount of medicine dispensed, as sensed by operations monitoring mechanism 28, can be flagged as an injected dose. On the other hand, where a pressure above the pressure threshold is not detected and/or other pressure metric(s) are not met prior to, during, for the duration of, at the conclusion of, and/or for a sufficient time during actuation of pen 20 to dispense medicine, the amount of medicine dispensed, as sensed by operations monitoring mechanism 28, can be flagged as a priming event.

Turning to FIGS. 6A and 6B, in aspects, a sensor 610 or 620 may be disposed on cartridge housing 23d or needle hub 29a, respectively. Sensors 610, 620 may be contact sensors, e.g., capacitive or resistive sensors, configured to sense contact with a user's skin; proximity sensors, e.g., hall effect sensors, mechanical switch, optical proximity sensors, etc., configured to sense proximity to an object, e.g., a user's skin; optical sensors configured to sense lighting and/or coloring, e.g., to determine the presence of a user's skin; and/or any other suitable sensors configured to sense contact with or close approximation to a user's skin. In this manner, sensors 610, 620 can sense insertion of hollow needle body 29b of needle 29 into the user's skin until the skin abuts or closely approximates cartridge housing 23d or needle hub 29a, respectively, thus indicating that pen 20 is being utilized to inject a volume of medicine, e.g., insulin, into the user, rather than for a priming event. With additional reference to FIGS. 3A and 3B, sensor 610 may be electrically connected, e.g., via wires (not shown) or other conductive structures extending along, within, or through cartridge housing 23d to contact(s) 320 of cartridge housing 23d which, in turn, are connected with contact(s) 310 of body 22 upon engagement of cartridge housing 23d with body 22, to thereby enable powering of sensor 610 and communication of pressure signals from sensor 610 to electronics unit 27 and/or operations monitoring mechanism 28. Sensor 620 may be connected in a similar manner with an additional set of contact connections wherein contacts 624 disposed on needle hub 29a connect with contacts 626 disposed on cartridge housing 23d to enable connection to electronics unit 27 and/or operations monitoring mechanism 28 to be utilized similarly as detailed above or in any other suitable manner. Other suitable sensor types and connections are also contemplated.

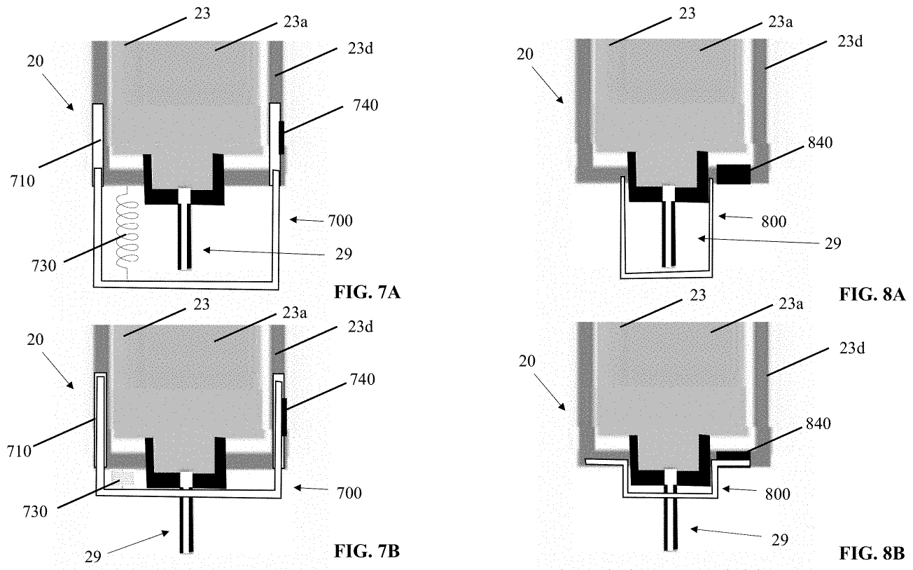

With reference to FIGS. 7A and 7B, in aspects, pen 20 includes a shroud 700 coupled to and extending from dispensing end of cartridge housing 23d to cover needle 29 in an extended position (FIG. 7A) of shroud 700. Shroud 700 may define a generally cylindrical configuration and may extend from and retract into a cylindrical track 710 defined within cartridge housing 23d (as shown) or may retract into a cylindrical space defined between medicine vial 23a of cartridge 23 and cartridge housing 23d. In either configuration, retraction of shroud 700 does not interfere with cartridge 23. Shroud 700 is biased, e.g., via a spring 730 or other suitable biasing member, towards an extended position (FIG. 7A), wherein shroud 700 substantially surrounds needle 29. Shroud 700 may be formed from a transparent material or any other suitable material.

As a user attempts to insert needle 29 into the user's skin, the user's skin contacts shroud 700 and, as needle 29 is inserted, urges shroud 700 against the bias of spring 730 such that shroud 700 is retracted at least partially into cylindrical track 710 to at least partially expose needle 29 (FIG. 7B) and enable insertion thereof. Upon sufficient retraction of shroud 700 into cylindrical track 710, a detector 740, e.g., a contact, proximity sensor, mechanical switch, or other suitable detector, detects the presence of shroud 700, thus indicating that an injection event, rather than a priming event, is occurring. With additional reference to FIGS. 3A and 3B, detector 740 may be electrically connected, e.g., via wires (not shown) or other conductive structures extending along, within, or through cartridge housing 23d to contact(s) 320 of cartridge housing 23d which, in turn, are connected with contact(s) 310 of body 22 upon engagement of cartridge housing 23d with body 22, to thereby enable powering of detector 740 and communication of a detection signal from detector 740 to electronics unit 27 and/or operations monitoring mechanism 28 to be utilized similarly as detailed above or in any other suitable manner.

FIGS. 8A and 8B illustrate another shroud 800 coupled to and extending from dispensing end of cartridge housing 23d and configured to move between an extended position (FIG. 8A), wherein shroud 800 substantially surrounds needle 29, and a retracted position (FIG. 8B), wherein shroud 800 is collapsed, folded, flexed, and/or otherwise retracted to at least partially expose needle 29 without interfering with cartridge 23. A detector 840 detects when shroud 800 is transitioned to the retracted position, thus indicating that an injection event, rather than a priming event, is occurring. Detector 840 may be configured to provide a detection signal similarly as detailed above with respect to detector 740 (FIGS. 7A and 7B). In aspects, shroud 800 may be biased towards the extended position as a result of the material(s) forming shroud 800, e.g., elastomeric, shape-memory, or other suitable materials, and/or via a separate biasing member (not shown; see spring 730 (FIGS. 7A and 7B)).

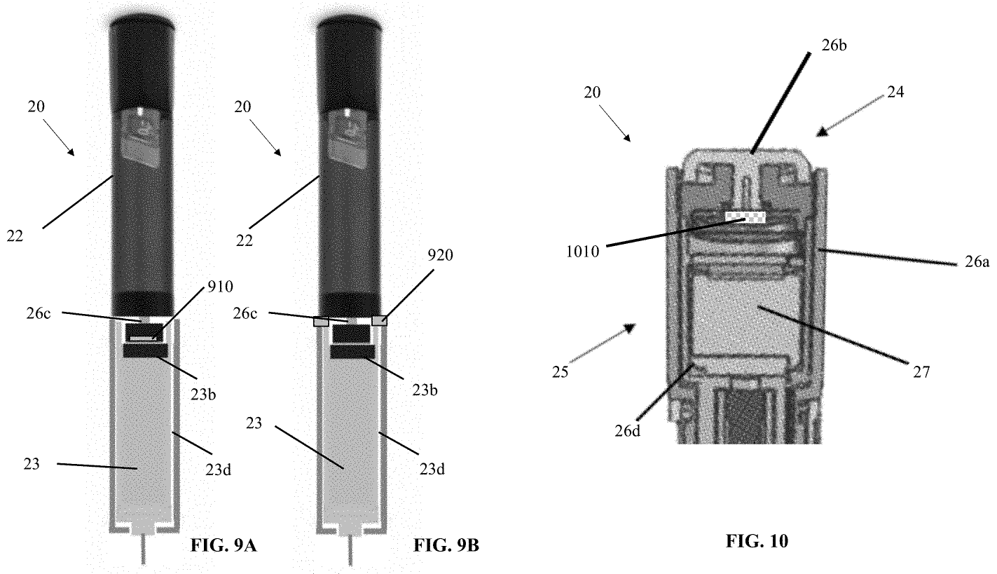

Referring to FIGS. 9A and 9B, in aspects, pen 20 includes a force sensor 910 or 920 disposed on or otherwise associated with drive screw 26c or disposed at the interface between body 22 of pen and cartridge 23 or cartridge housing 26d, respectively. Force sensor 910 may be a torque sensor, a linear force sensor, a pressure sensor (such as a pressure transducer), etc. Force sensor 920 may be a linear force sensor, a pressure sensor (such as a pressure transducer), etc. Force sensor 910, more specifically, is configured to distinguish between the force required to move drive screw 26c to thereby urge piston 23b to dispense medicine freely into the air as compared to the force required to move drive screw 26c to thereby urge piston 23b to inject medicine into the user. Force sensor 920, more specifically, is configured to sense a force at the interface between body 22 of pen and cartridge 23 or cartridge housing 26d and compare the same to a force threshold (or force profile, force trend, etc.) indicating a resistive force of the user's skin and/or tissue against cartridge 23 or cartridge housing 26d during injection. Thus, force sensors 910, 920 enable distinguishing between a priming event and an injection event, which can then be communicated from force sensors 910, 920 to electronics unit 27 and/or operations monitoring mechanism 28 (see FIGS. 2B and 3B) to be utilized similarly as detailed above or in any other suitable manner.

As shown in FIG. 10, in aspects, pen 20 may include a force sensor 1010, e.g., a linear force sensor, a pressure sensor (such as a pressure transducer), etc., associated with, e.g., directly or indirectly contacted by, dose dispensing button 26b of dose dispensing mechanism 24 when dose dispensing button 26b is actuated to begin dispensing. Force sensor 1010 is thus capable of sensing a force required to actuate dose dispensing button 26b and compare the same to a force threshold (or force profile, force trend, etc.) indicating a resistive force of the user's skin and/or tissue against injection. Thus, force sensor 1010 enable distinguishing between a priming event and an injection event; the same may be communicated from force sensor 1010 to electronics unit 27 and/or operations monitoring mechanism 28 (see FIGS. 2B and 3B) to be utilized similarly as detailed above or in any other suitable manner.

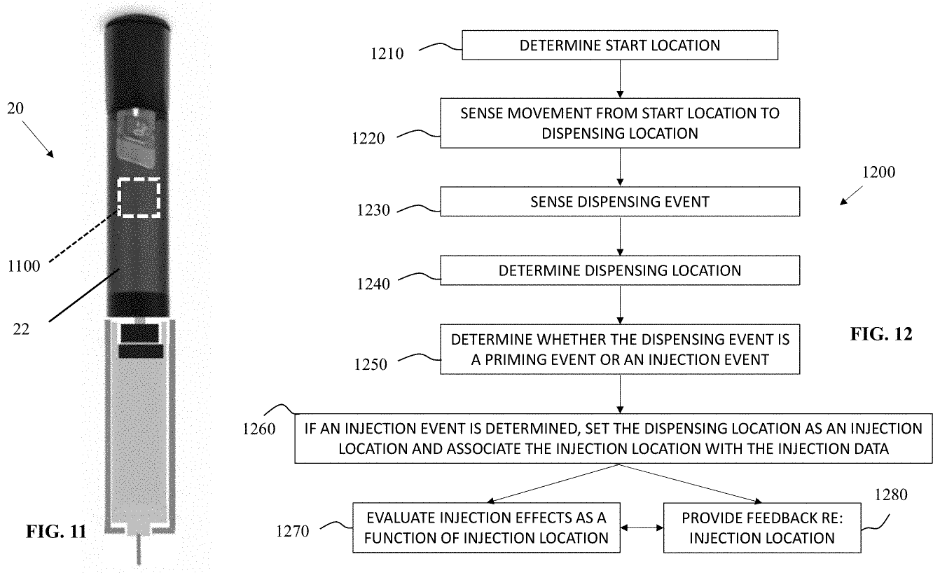

Turning to FIGS. 11 and 12, in aspects, pen 20 further includes one or more motion sensors 1100, e.g., disposed within body 22 or otherwise positioned on or in pen 20. In configurations where multiple motion sensors 1100 are provided, the motion sensors 1100 may be disposed at different locations, e.g., radial and/or longitudinal positions along body 22 or otherwise within or on pen 20, and may be similar or different from one another. Each of the one or more motion sensors 1100 may include, for example, a 3-axis motion sensor (e.g., a 3-axis accelerometer, a 3-axis gyroscope, a 3-axis magnetometer, or other suitable 3-axis motion detector or combination of motion detectors to provide 3-axis motion sensing); a 6-axis motion sensor (e.g., any two of the above or other suitable motion detectors or combinations thereof); a 9-axis motion sensor (e.g., any three of the above or other suitable motion detectors or combinations thereof); or any other suitable accelerometer, gyroscope, magnetometer, GPS sensor, other motion detectors, combinations thereof, etc. configured to sense position, translational movement (magnitude and/or direction), rotational movement (magnitude and/or direction), orientation, speed, acceleration, etc.

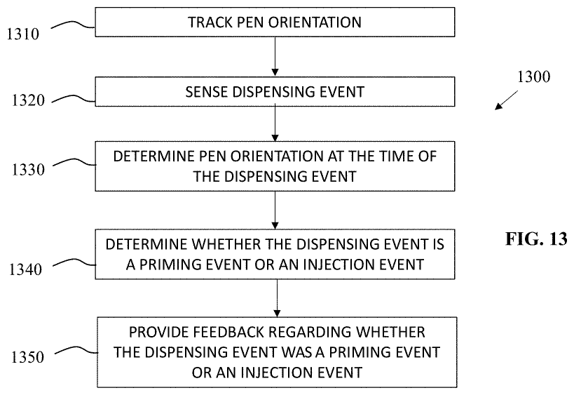

In aspects, for example, orientation data may be utilized to distinguish a priming orientation during dispensing, e.g., vertical or near vertical (such as within 15% of vertical), from an injection orientation during dispensing, e.g., horizontal or near horizontal (such as within 15% of horizontal), and/or distinguish based on orientation over time, e.g., a sequence of orientations indicating priming versus a sequence of orientations indicating injection. More specifically, and with momentary reference to FIG. 13, in conjunction with FIGS. 10 and 11, in aspects, a method of distinguishing a priming event from an injection event is shown as method 1300. Method 1300 starts at 1310 wherein the orientation of the pen is tracked during movement of the pen, e.g., via the one or more motion sensors 1100. In other aspects, orientation tracking is not provided. When a dispensing event occurs, the dispensing event is sensed at 1320, e.g., via operations monitoring mechanism 28 (FIGS. 2B and 3B) determining that a dispensing event has been initiated, has occurred, and/or determining (or enabling determination of) an amount of medicine dispensed. The pen orientation at the time of the dispensing event (e.g., at initiation of the dispensing event, at the conclusion of the dispensing event, throughout the dispensing event, etc.) is determined at 1330, e.g., using the motion sensor data together with the dispensing event data and, in aspects, timestamp or other temporal data. At least in aspects where orientation tracking is not utilized, an absolute orientation may be determined at the time of the dispensing event. On the other hand, relative orientation tracking may be implemented based on the orientation tracking data. Regardless of the manner in which the orientation at the time of the dispensing even is determined, it is then determined, at 1340, based upon the determined pen orientation at the time of the dispensing event, whether the dispensing event is a priming event or an injection event. For example, where the orientation at the time of the dispensing event is vertical or near vertical (such as within 15% of vertical) with the needle end of the pen facing upwardly, a priming event may be determined. In all other orientations, or in select other orientations, e.g., where the orientation at the time of the dispensing event is horizontal or near horizontal (such as within 15% of horizontal) and/or where the orientation at the time of the dispensing event is vertical or near vertical (such as within 15% of vertical) with the needle end of the pen facing downwardly, an injection event may be determined. Other suitable orientations may also be utilized to determine priming versus injection. Further, in aspects, some orientations may enable automatic determination of a priming event versus an injection event (such as those noted above) while other orientations require user confirmation of a determined probable priming event or injection event, in order to inhibit incorrect categorization. Alternatively or additional, other suitable user input data or sensed data (such as dispensed amount, other movement data, historical data relating to confirmed priming and/or injection events, etc.) may be utilized to facilitate distinguishing between a priming event and an injection event, in all cases or at least in cases where the orientation sensing is inconclusive as to whether a priming event or an injection even occurred. In aspects, priming orientations and/or injection orientations may be learned and/or set by the user to facilitate personalized determination of whether a user conducted a priming event or an injection event based on the orientation, location, etc. the user typically holds the pen for priming and/or injection events. At 1350, feedback is provided regarding whether the dispensing event was a priming event or an injection event. The feedback may be saved in electronics unit 27 and/or communicated to health management application 40 (FIG. 1A) of smartphone 30 (FIGS. 1A-1C). The feedback, indicating whether the dispensing event was determined to be an injection event or a priming event, may be utilized, to record an injected dose (if an injection event), update a dose calculation, update insulin on board (if an injection event), update insulin remaining in the cartridge, clear or update notifications, etc.

Referring again to FIGS. 11 and 12, regardless of the particular use of the one or more motion sensors 1100, the one or more motion sensors 1100 may be configured for use in a power-save or low-power configuration, e.g., such that motion sensors 1100 are not continuously active when a user is carrying pen 20, moving pen 20 (with no intention to dispense), etc., thereby preserving battery life. Motion sensor(s) 1100 may be configured to “wake-up” upon detection of motion; however, this alone would not prevent active motion tracking when pen 20 is being moved without the intention of dispensing. Thus, motion sensor(s) 1100 may be configured to “wake-up” in response to sensing a recognized movement pattern, based on feedback from another sensor or device, e.g., an audible or other sensor that recognizes removal of cap 21 (FIG. 1C), a communication from smartphone 30 (FIGS. 1A-1C) that health management application 40 (FIG. 1A) was recently utilized, movement of dose knob 26ba, combinations thereof, or in any other suitable matter.

In addition or as an alternative to motion sensing within pen 20 itself, motion sensing information pertaining to the user may be provided by smartphone 30 (FIGS. 1A-1C) and/or other connected device(s), e.g., a smart watch or fitness tracker. Such motion sensor may be utilized, for example, to confirm motion sensing data obtained from pen 20 and/or in any other suitable manner. With additional reference to FIGS. 1A-1C, regardless of the particular source or combination of sources, sensed motion data may be provided, e.g., from electronics unit 27 (which is connected to the one or more motion sensors 1100) of pen 20, to health management application 40 running on smartphone 30 to enable processing of the sensed motion data together with dispensing data from pen 20 to distinguish whether the dispensing event was a priming event or an injection event. For example, the amount of movement, direction of movement, orientation of pen 20 during dispensing, etc. may be utilized to categorize a dispensing event as a priming event or an injection event.

Continuing with reference to FIGS. 1A-1C, 11, and 12, the motion sensing information provided by the one or more motion sensors 1100 of pen 20, smartphone 30, and/or other connected device(s), may additionally or alternatively be utilized to determine an injection location, relative to the user's body, for an injection event and to provide insight based thereon, e.g., as detailed below with respect to method 1200. For example, as another example, based on historical data, input data, or other suitable information, a reference location such as a start location, e.g., start position and/or orientation, may be determined, as indicated at step 1210. The start location may be, for example, the position of pen 20 relative to the user's body where the user removes cap 21 (FIG. 1B) from pen 20, the position of pen 20 relative to the user's body where the user sets the dose, or any other reference position, e.g., the position and/or orientation of pen 20 relative to the floor, a table surface, etc. With the start location determined at step 1210, the one or more sensor 1100 can be utilized to sense movement of pen 20 from the start location to a dispensing location, as indicated at step 1220. The dispensing location may be determined as the location where movement stops and/or when another activity begins, e.g., dispensing is initiated. As indicated at step 1230, a dispensing event is sensed, e.g., via operations monitoring mechanism 28 (FIGS. 2B and 3B) determining that a dispensing event has been initiated, has occurred, and/or determining (or enabling determination of) an amount of medicine dispensed.

Prior to, contemporaneously with, or after sensing the dispensing event, the dispensing location may be determined, as indicated at step 1240. The dispensing location may be a location relative to the user's body, e.g., away from the user's body, sufficiently approximated relative to the user's body, or on a particular location on the user's body such as at the user's left arm, right arm, left side of torso, center of torso, right side of torso, right upper leg, left upper leg, etc., or any other suitable identifiable location. The dispensing location may be determined based on motion sensing information provided by the one or more motion sensors 1100 of pen 20, smartphone 30, and/or other connected device(s), to enable tracking movement of the pen 20 from the start location to the dispensing location.

Based at least on the dispensing location determined at step 1240 (and, in aspects, also based on the dispensing event sensed at step 1230), it can be determined whether the dispensing event was a priming event or an injection event at step 1250. For example, if the dispensing location is determined to be away from the user's body, the dispensing event can be classified as a priming event. On the other hand, if the dispensing location is determined to be sufficiently approximated relative to or on a particular part of the user's body (e.g., where injections typically occur), the dispensing event can be classified as an injection event.

Continuing to step 1260 of method 1200, if the dispensing event is determined to be an injection event, the dispensing location can be associated, e.g., stored together with, the injection data (e.g., the information from sensing the dispensing event in step 1230), in electronics unit 27 and/or on health management application 40 (FIG. 1A) of smartphone 30 (FIGS. 1A-1C) as an injection location. The injection data and associated injection location data can then be utilized to evaluate injection effects as a function of injection location, as indicated at step 1270, and/or to provide feedback regarding injection location, as indicated at step 1280. With respect to evaluating injection effects as a function of injection location, health management application 40 (FIG. 1A) of smartphone 30 (FIGS. 1A-1C) may track insulin effects over time, e.g., amount and/or rate of insulin absorbed, based upon the injection location in order to, for example, recommend to the user an optimal injection location to maximize insulin absorption and/or for other purposes. With respect to providing feedback regarding injection location, health management application 40 (FIG. 1A) of smartphone 30 (FIGS. 1A-1C) may track the injection locations over time to recommend that the user move the injection location, e.g., where injections are repeated in the same or close locations, in order to enable that injection site to heal or for other purposes.

In aspects, the one or more motion sensors 1100 may be utilized to enable detection of when pen 20 has been dropped and/or when any other high-impact event has occurred. Where a drop or high-impact event is detected, pen 20 may communicate with health management application 40 (FIG. 1A) of smartphone 30 (FIGS. 1A-1C) to provide a warning and/or instructions to the user. The warning and/or instruction may include, for example, a warning and/or instructions to check the functionality of pen 20, inspect pen 20 for damaged and/or misaligned components, perform a functionality test of pen 20, dis-assemble and re-assemble pen 20, etc. Drop or high-impact event information may also be stored in the memory of pen 20 and/or smartphone 30 (FIGS. 1A-1C), and/or may be communicated to another connected device together with identifying information of pen 20 for use in, for example, considering warranty claims, to facilitate diagnostics of a malfunctioning pen, and/or for other purposes.

In additional or alternative aspects, the one or more motion sensors 1100 may be utilized to enable detection of when pen 20 is manipulated and/or actuated in an erratic manner such as, for example, where pen 20 is violently shaken, when dose knob 26a is repeatedly ratcheted back and forth, etc. Where such an event is detected, pen 20 may communicate with health management application 40 (FIG. 1A) of smartphone 30 (FIGS. 1A-1C) to provide a warning and/or instructions to the user, such as those detailed above or any other suitable warning and/or instructions, e.g., indicating to the user to stop the behavior at issue. Such information may also be communicated to another connected device together with identifying information of pen 20 for use in, for example, considering warranty claims, to facilitate diagnostics of a malfunctioning pen, and/or for other purposes.

Further, the one or more motion sensors 1100 may be utilized to enable detection of when medicine cartridge 23 (and/or a different medicine cartridge) is inserted and/or removed, e.g., based on sensing tactile feedback resulting from the physical insertion and/or removal, and/or in any other suitable manner. Alternatively or additionally, using motion sensors 1110 and/or separate device(s), gesture recognition may be utilized to enable detection of when the medicine cartridge is inserted and/or removed. Where it is determined that the medicine cartridge is removed and a new, different, or re-filled medicine cartridge is inserted, pen 20 may store information and/or communication with health management application 40 (FIG. 1A) of smartphone 30 (FIGS. 1A-1C) to warn the user that they should prime the newly-installed cartridge before an injection, to indicate that a subsequent dispensing event may be a priming event (based on this information alone or in connection with additional information, e.g., where less than a pre-determined amount of medicine is dispensed in the subsequent dispensing event), and/or to warn the user to confirm that the correct type of insulin is installed. Such information may also be communicated to another connected device for use in, for example, ensuring patient compliance with the prescribed regime and/or for other purposes.

Any or all of the prime versus injection distinctions detailed above may further be confirmed or revised, e.g., via health management application 40 (FIG. 1A) based on glucose response data, e.g., amount and/or rate of insulin absorbed, insulin on board, etc., and/or other physiological parameter data. For example, health management application 40 (FIG. 1A) may confirm that, based on the categorization of dispensing events as priming or injection events, the glucose response data is within an expected range, e.g., based on historical data. If the data is not determined to be within an expected range, this may indicate that a dispensing event was miscategorized. Health management application 40 (FIG. 1A) may, in response to determining the data is not within the expected range, request that the user manually confirm the dispensing events as priming or injection, or may re-categorize the most likely incorrect events to see if, based on the revised categorization, the data is within the revised expected range.

Further aspects, features, and details of mechanical, hardware, and/or software-implemented configurations for distinguishing priming events from injection events can be found in commonly-owned International Patent Application Publication No. WO 2020/214981, titled “Prime Differentiation in Medication Delivery System” and filed on Apr. 17, 2020, the entire contents of which are hereby incorporated herein by reference. The aspects and features of the present disclosure may be utilized, in whole or in part, together with those, in whole or in part, of the WO 2020/214981 Publication in any suitable combination to facilitate prime differentiation and/or for other purposes.

The various aspects and features disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described functional and/or operational aspects may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing unit” as used herein may refer to any of the foregoing structure or any other physical structure suitable for implementation of the described techniques. Also, the techniques could be fully implemented in one or more circuits or logic elements.

While several aspects of the present disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. A method for medicine administration and tracking, comprising:
tracking movement of an injection pen from a start location to a dispensing location;
detecting a dispensing event of medicine dispensed from the injection pen at the dispensing location;
determining a location of the dispensing location relative to a user's body;
associating information relating to the detected dispensing event with information relating to the determined location; and
providing feedback based upon the detected dispensing event information and the determined location information.

2. The method according to claim 1, wherein the feedback includes a recommended injection location.

3. The method according to claim 2, wherein the recommended injection location is determined based upon evaluating at least one physiological parameter associated with the dispensing event at the determined location.

4. The method according to claim 3, wherein the physiological parameter is at least one of insulin absorbed or an insulin absorption rate.

5. The method according to claim 1, wherein the feedback includes a recommendation to change an injection location.

6. The method according to claim 5, wherein the recommendation to change the injection location is determined based upon evaluating a number of dispensing events occurring at the determined location.

7. The method according to claim 1, further comprising, determining whether the dispensing event is a priming event or an injection event based upon the determined location.

8. The method according to claim 7, wherein the associating and providing feedback are only performed in a case where it is determined that the dispensing event is a priming event.

9. A medicine administration and tracking system, comprising:
an injection pen configured to dispense medicine, the injection pen including at least one sensor configured to sense a dispensing event of medicine dispensed from the pen, at least one motion sensor configured to provide information regarding movement of the injection pen, and an electronic unit configured to transmit information regarding the dispensing event and the information regarding movement of the injection pen; and
a computing device configured to receive the information regarding the dispensing event and the information regarding movement of the injection pen, the computing device including a processor and a memory storing a health management application that, when executed by the processor, causes the processor to:
track movement of the injection pen from a start location to a dispensing location based upon the information regarding movement of the injection pen, wherein the dispensing location corresponds to the position where the dispensing event occurred;
determine a location of the dispensing location relative to a user's body;
associate the information regarding the dispensing event with information relating to the determined location; and
provide feedback based upon the dispensing event information and the determined location information.

10. The system according to claim 9, wherein the feedback includes a recommended injection location determined based upon evaluating at least one physiological parameter associated with the dispensing event at the determined location.

11. The system according to claim 10, wherein the physiological parameter is at least one of insulin absorbed or an insulin absorption rate.

12. The system according to claim 9, wherein the feedback includes a recommendation to change an injection location determined based upon evaluating a number of dispensing events occurring at the determined location.

13. The system according to claim 9, wherein the processor is further caused to determine whether the dispensing event is a priming event or an injection event based upon the determined location and associate the information and provide the feedback only in a case where it is determined that the dispensing event is a priming event.

14. A medicine injection pen, comprising:
a body;
a drive member disposed within the body and configured to move relative to the body upon actuation of the drive member;
a cartridge housing engaged with the body and configured to retain a medicine cartridge therein, the medicine cartridge including a medicine vial configured to retain a liquid medicine therein, a needle disposed at a dispensing end of the medicine vial, and a piston configured to slide within the medicine vial, wherein the drive member is configured to move relative to the body upon actuation thereof to urge the piston to slide within the medicine vial to thereby dispense at least some of the liquid medicine through the needle;
at least one sensor configured to sense movement of the drive member relative to the body upon actuation of the drive member to enable determination of an amount of the liquid medicine dispensed based on the sensed movement; and
a thin-film pressure sensor disposed on one of: a portion of the cartridge housing, a portion of the medicine vial, or a portion of needle and configured to detect pressure applied by a user's skin to thereby enable determination of whether the amount of the liquid medicine dispensed was dispensed as a priming event or an injection event.

15. The medicine injection pen according to claim 14, wherein the thin-film pressure sensor defines a ring-shaped configuration.

16. The medicine injection pen according to claim 14, wherein the thin-film pressure sensor is disposed on an exterior end face of the cartridge housing.

17. The medicine injection pen according to claim 14, wherein the thin-film pressure sensor is disposed on an exterior end face of a hub of the needle.

18. The medicine injection pen according to claim 17, further comprising mating contacts disposed on the hub of the needle and the cartridge housing to electrically connect the thin-film pressure sensor to an electronics unit disposed within the body.

19. The medicine injection pen according to claim 14, wherein the cartridge housing is releasably engaged with the body and wherein mating contacts are disposed on the cartridge housing and the body to electrically connect the thin-film pressure sensor to an electronics unit disposed within the body.

20. A medicine administration and tracking system, comprising:
an injection pen configured to dispense medicine, the injection pen including at least one sensor configured to sense a dispensing event of medicine dispensed from the pen, at least one motion sensor configured to provide information regarding an orientation of the injection pen, and an electronic unit configured to transmit information regarding the dispensing event and the information regarding the orientation of the injection pen; and
a computing device configured to receive the information regarding the dispensing event and the information regarding the orientation of the injection pen, the computing device including a processor and a memory storing a health management application that, when executed by the processor, causes the processor to:
determine, based on the information regarding the dispensing event, occurrence of the dispensing event;
determine, based on the information regarding the orientation of the injection pen, the orientation of the injection pen at a time of the dispensing event;
determine, based on the orientation of the injection pen at the time of the dispensing event, whether the dispensing event is a priming event or an injection event; and
provide feedback regarding whether the dispensing event was as priming event or an injection event.
